(19) United States
(12) Patent Application Publication
(10) Pub. No.: US 2008/0317849 A1
(43) Pub. Date: Dec. 25, 2008

(54) Method for Improving the Medical Treatment of Pain

(76) Inventors: Stephan Christgau, Gentofte (DK); Christian Hansen, Vedbaek (DK); Henrik Nilsson, Copenhagen K (DK)

Correspondence Address: JONES DAY, 222 EAST 41ST ST, NEW YORK, NY, 10017, US

(21) Appl. No.: 11/629,612

(22) PCT Filed: Jun. 17, 2005
(86) PCT No.: PCT/DK05/00401

§ 371 Date: Mar. 4, 2008

(30) Foreign Application Priority Data
Jun. 17, 2004 (DK) PA2004-00947

(51) Int. Cl. A61K 31/192; A61K 31/185; A61K 31/415; A61K 9/20; A61P 25/00; A61P 19/02; A61K 31/60

(52) U.S. Cl. 424/464; 514/574; 514/569; 514/406; 514/163

(57) ABSTRACT

Methods for improving pain management in a mammal, the methods comprising administering a combination of a strontium-containing compound and a second therapeutically and/or prophylactically active substance selected from the group consisting of analgesic agents, anti-inflammatory agents and palliative agents to the mammal. Pharmaceutical compositions for use in such methods, comprising a strontium-containing compound and a second therapeutically and/or prophylactically active substance selected from the group consisting of analgesic agents, anti-inflammatory agents and palliative agents.

### FIELD OF THE INVENTION

The present invention relates to methods for improving pain management in a mammal, the methods comprising administering a combination of a strontium-containing compound and a second therapeutically and/or prophylactically active substance selected from the group consisting of analgesic agents, anti-inflammatory agents and palliative agents to the mammal. The invention also relates to pharmaceutical compositions for use in such methods.

### BACKGROUND OF THE INVENTION

One of the most common symptoms associated with both chronic and acute diseases, disorders, trauma and medical conditions, is the presence of pain. Pain may be the symptom responsible for most physician visits, and pain is fundamental to medicine and in defining the well-being of individuals. Although all human beings will experience pain at some level in many different situations, pain remains extremely difficult to define and quantify and the etiology of pain remains elusive. Aside from the physiological processes of pain induction, many psychological and psychosocial factors are related to adjustment to persistent pain. Many of such poorly defined and quantified factors are associated with increased sensation of pain and poorer adjustment to pain (i.e., pain catastrophizing, pain-related anxiety and fear of pain, and helplessness). Other psychological and psychosocial factors are associated with decreased pain and improved adjustment to pain (i.e., self-efficacy, belonging to the male gender, pain coping strategies, readiness to change, and acceptance). In the clinical management of pain, medications able to combat physiological processes involved in pain sensation either at peripheral sites or in the central nervous system (CNS) plays a central role, and analgesic/palliative medications remains some of the most prescribed drugs in use today. However, even with recent advances in the development of new palliative and analgesic agents, the medical interventions available today for the treatment of pain remains associated with substantial side effects.

Pain can present itself in many ways, and is associated with a multitude of physiological reactions and medical conditions, many of which seems to be associated with an inflammatory response and/or cytokines and signaling molecules such as prostaglandins, leukotrienes, TNF-α and substance P involved in inflammatory responses and soft tissue reactions to noxious stimuli. In management of pain it is often useful to distinguish between chronic pain and acute pain. The later form of pain can usually be associated with a stimulus causing tissue injury and release of intra- as well and inter-cellular signaling molecules responsible for initiating the sensation of pain but also the repair mechanisms. Chronic pain can in many situations be difficult to associate with specific pathological mechanisms at the tissue level, but in many chronic conditions associated with pain, such as rheumatoid arthritis and osteoarthritis, evidence of systemic elevation in inflammatory processes and inflammation related cytokines can be detected, and at least in part this can explain the pain of the patient. However, it must be pointed out that many conditions of severe chronic pain exist, which appear to be completely maladaptive and not related to any ongoing noxious stimuli, i.e. in conditions such as fibromyalgia.

A number of drugs have been developed to treat pain symptoms. In very broad terms, most drugs used in clinical practice today can be divided in two classes, opioids and non-steroidal anti inflammatory drugs (NSAIDs). Opioids target primarily receptors in the central nervous system (CNS) responsible for the sensation of pain, whereas NSAIDs comprise a heterogeneous group of compounds with an ability to reduce inflammatory signaling molecules such as prostaglandin synthesis and cyclo-oxygenase enzymes. Both of these drug types are associated with significant side effects such as a development of drug dependency and abuse (opioids) and gastrointestinal and cardiovascular complications (NSAIDS). Conventional non-steroidal anti-inflammatory drugs (NSAIDs) e.g. ibuprofen, inhibit both the isoforms of the cyclo-oxygenase enzyme. The enzyme cyclo-oxygenase (COX) exists as two distinct isoforms. COX-1 is constitutively expressed as a ‘housekeeping’ enzyme in nearly all cells and tissues, and mediates a number of essential physiological responses (e.g. cytoprotection of the stomach, and platelet aggregation). On the other hand, COX-2, expressed by cells involved in inflammation (e.g. activated macrophages, monocytes, synoviocytes), has emerged as the isoform that is primarily responsible for the synthesis of prostanoids of which prostaglandins are the most prominent group of compounds. These molecules are involved in acute and chronic inflammatory states and their production locally or systemically induce a range of physiological reactions ranging from activation of sensory neurons to initiation of tissue repair as well as tissue catabolism. Consequently, the hypothesis that selective inhibition of COX-2 might have therapeutic actions similar to those of non-steroidal anti-inflammatory drugs, but without causing gastrointestinal side effects, was the rationale for the development of selective inhibitors of the COX-2 isoenzyme. Selective COX-2 inhibitors currently used in the clinic are the sulphonamides celecoxib and valdecoxib (parecoxib is a prodrug of valdecoxib), as well as the methylsulphones rofecoxib and etoricoxib. Furthermore, the phenylacetic acid derivatives lumiracoxib and deracoxib has recently been introduced into clinical practice. A number of other COX-2 specific inhibitors have been described in the literature and several are in different stages of development.

Of great importance for the clinical use of NSAIDs and COX-2, is the well supported notion that inhibition of COX-1 is thought to be principally responsible for the gastrointestinal adverse effects of NSAIDs. As a result COX-2 inhibitors e.g. rofecoxib, celecoxib, valdecoxib lumiracoxib and deracoxib as listed above is believed to be associated with significantly less GI side effects, and in fact this issue of reduced GI toxicity provided the main impetus for the development of this new drug class. However, there is some uncertainty regarding the cardiovascular and renal effects of the COX-2 selective inhibitors, and these drugs are still associated with a significantly increased risk of GI side effects.

In the management of acute pain, the ability to prevent the onset of pain, lessen its intensity, and interfere with the development of sensitization contributing to hyperalgesia for days following traumatic pain inducing events such as a surgical procedure or a major traumatic event can greatly benefit the patient, rather than postoperative attempts to decrease pain after it has reached full intensity. In situations where pain can be anticipated, i.e. in a surgical procedure, the NSAID may be optimized by preoperative administration and continuing to dose the NSAID on a regular schedule to minimize pain and inflammation. Patients benefit from receiving optimal NSAID doses, and in some cases very high doses of these palliative agents are required to efficiently relieve the pain. In conditions of chronic pain, the dosing of palliative agents are of paramount importance, and as many These agents are effective and reduce the need for opioids, but they are associated with a number of deleterious side-effects, of which the well documented gastrointestinal (GI) irritation is the most serious. Traditional NSAIDs are also associated with reduced platelet function and thus an increased risk of cardiovascular events. Opioids are often used with great caution due to the fear of exposing the patient to risk of developing drug dependency. Thus, due to the anticipated side effects of current palliative medications, patients in need of analgesic treatments often receive insufficient doses and/or length of treatment of the palliative agent(s). Therefore there is a pressing need for methods and agents that can improve pain treatment.

In the clinical development of these compounds their palliative effects have been the primary endpoint of the investigations. Generally all the COX-2 inhibitors introduced in the clinical practice today have been demonstrated to provide as effective pain relief as conventional NSAIDs such as naproxen, oxycodone and acetaminophen. Of particular relevance it has been demonstrated that many COX-2 inhibitors have an opioid-sparing effects, i.e. they enable a reduction or complete withdrawal of opioid use in palliative treatments of the patients. This has been especially demonstrated for diseases and disorders of the articular joints and muscoskeletal system. As an example consistent use of a COX-2 inhibitor after hip or knee arthroplasty can substantially reduce or avoid the need for strong palliative interventions such as opioid treatment.

However, contrary to what was originally believed when this new class of drugs were developed, COX-2 inhibitors is still associated with an increased prevalence of GI side effects compared to placebo treatment. The incidence of gastroduodenal ulcers in COX-2 inhibitor treated patients is generally lower than with nonselective NSAIDs (i.e. NSAIDs not specifically developed as selective COX-2 inhibitors), but still significantly higher that in placebo treated individuals. With concomitant aspirin, the ulcer rate in COX-2 recipients is increased significantly, but still lower than that in recipients of aspirin plus nonselective NSAIDs. Thus in spite of the potential promise held by the COX-2 selective anti-inflammatory agents there is still an unmet medical need to improve the palliative treatments and in particular there is a need for medical treatments that can reduce the incidence of GI side effects.

### DESCRIPTION OF THE INVENTION

We have found that the coadministration by the oral route of a cyclo-oxygenase (COX) inhibitor, and in particular an inhibitor of COX-1 and a strontium containing compound such as an inorganic or organic strontium salt can reduce the GI side effect associated with administration of the COX inhibitor alone while improving the palliative efficacy of the compound(s).

Accordingly the present invention comprises methods, pharmaceutical formulations kits and medical treatments where a COX inhibitor and a strontium containing compound is administered in combination to a subject in need thereof.

In a further embodiment of the invention, we have found that a strontium containing compound not only enable an improvement in palliative treatment when administered in combination with a COX-2 specific inhibitor. The beneficial effects of coadministration of a strontium compound applies equally well for therapies with other palliative treatments pharmaceutical drug classes comprising NSAIDs, COX-2 inhibitors, COX-3 inhibitors, iNOS inhibitors, PAR2 receptor antagonists, neuroleptic agents, opioids, N-acetylcholine receptor agonists, glycine antagonists, vanilloid receptor antagonists, neurokinin antagonists calcitonine gene-related peptide antagonists and Cyclooxygenase (COX)-inhibiting nitric oxide donators (ClNOD).

A particular useful embodiment of the present invention is to use a combination product containing a strontium compound and a second palliative/analgesic agent in mammal such as a human suffering from a muscoskeletal disorder such as OA, RA or osteoporosis. In such applications, the dual action of strontium on both the pain and symptoms of the disease as well as the underlying progression of structural deterioration such as elevated cartilage degradation, elevated bone resorption and/or decreased bone formation is particularly useful. In addition to the effect on soft tissue pains of certain strontium products as disclosed in patent WO 03028742 we have found that strontium is active also on the underlying processes of structural deterioration in muscoskeletal diseases thereby providing the basis for a sustained effect on the diseases as well as both a prophylactic and therapeutic clinical use of combination products according to the present invention.

A central aspect of this invention is the use of an orally administered strontium containing compound for improving the management of pain and/or palliative treatment associated with a of acute or chronic conditions involving elevated sensation of pain either locally or systemically. In one aspect of the invention this comprise a method of alleviating pain in an animal including a mammal, comprising administering to the animal a pain alleviating effective amount of a means for alleviating pain in an animal in admixture with a pharmaceutically acceptable carrier, diluent, or excipient.

Another aspect is any one of the above methods of alleviating pain, wherein the pain is

osteoarthritic pain,
rheumatoid arthritic pain,
juvenile chronic arthritis associated pain,
juvenile idiopathic arthritis associated pain,
Spondyloarthropathies (such as ankylosing spondylitis (Mb Bechterew) and reactive arthritis (Reiter's syndrome)) associated pain,
pain associated with psoriatic arthritis,
gout pain,
pain associated with pseudogout (pyrophosphate arthritis),
pain associated with systemic lupus erythematosus (SLE),
pain associated with systemic sclerosis (scleroderma),
pain associated with Behçet's disease,
pain associated with relapsing polychondritis,
pain associated with adult Still's disease,
pain associated with transient regional osteoporosis,
pain associated with neuropathic arthropathy,
pain associated with sarcoidosis,
arthritic pain,
rheumatic pain,
joint pain,
osteoarthritic joint pain,
rheumatoid arthritic joint pain,
juvenile chronic arthritis associated joint pain,
juvenile idiopathic arthritis associated joint pain,
Spondyloarthropathies (such as ankylosing spondylitis (Mb Bechterew) and reactive arthritis (Reiter's syndrome)) associated joint pain,
joint pain associated with psoriatic arthritis,
gout joint pain,
joint pain associated with pseudogout (pyrophosphate arthritis),
joint pain associated with systemic lupus erythematosus (SLE),
joint pain associated with systemic sclerosis (scleroderma),
joint pain associated with Behçet's disease,
joint pain associated with relapsing polychondritis,
joint pain associated with adult Still's disease,
joint pain associated with transient regional osteoporosis,
joint pain associated with neuropathic arthropathy,
joint pain associated with sarcoidosis,
arthritic joint pain,
rheumatic joint pain,
acute pain,
acute joint pain,
chronic pain,
chronic joint pain,
inflammatory pain,
inflammatory joint pain,
mechanical pain,
mechanical joint pain,
pain associated with the fibromyalgia syndrome (FMS),
pain associated with polymyalgia rheumatica,
monarticular joint pain,
polyarticular joint pain,
nociceptiv pain,
neuropathic pain,
psychogenous pain.
pain of unknown etiology,
pain mediated by IL-6, IL-6 soluble receptor, or IL-6 receptor, pain associated with a surgical procedure in a patient with a clinical diagnosis of OA, dental pain,
pain associated with a surgical procedure and or other medical intervention,
bone cancer pain,
neuropathic pain,
pain associated with migraine,
pain like static allodynia,
pain like dynamic allodynia,
pain associated with Crohn's disease
headache pain and/or
pain associated with completion of a large number of patent applications within a limited interval of time.

Another aspect is any one of the above methods of alleviating pain other than joint pain, osteoarthritic pain, rheumatoid arthritic pain, and inflammatory joint pain, wherein the pain is pain mediated by IL-6, IL-6 soluble receptor, or IL-6 receptor.

A further aspect of this invention is any one of the above methods of alleviating pain, wherein the pain is mediated by a protein or protein and its receptor selected from: oncostatin-M, oncostatin-M and oncostatin-M receptor, leukemia inhibitor factor (“LIF”), LIF and leukemia inhibitor factor receptor (“LIFR”), interleukin-1 (“IL-1”), and interleukin-1 receptor (“IL1 R”).

A still further aspect is any one of the above methods of alleviating pain other than joint pain, osteoarthritic pain, rheumatoid arthritic pain, and inflammatory joint pain, wherein the pain is pain mediated by endothelin

A strontium salt for a use according to the invention should preferentially be water soluble, and in one embodiment of the present invention, the pH of an aqueous solution of a strontium salt according to the invention has a pH of more than 10. Di-anionic amino-acid salts of strontium, such as strontium aspartate and strontium glutamate but also dicarboxylic anion salts of strontium such as strontium malonate, strontium succinate, strontium pyruvate, strontium fumarate, strontium maleate and strontium oxalate may be especially suited for a pharmaceutical use according to the invention.

Other specific strontium salts which may be used to carry out a medical treatment according to the present inventions will contain an anion with a suitable pharmacologic action such as: strontium L-ascorbate, strontium acetyl-salicylate, strontium salicylate, strontium alendronate, strontium ibandronate, strontium salts of propionic acids such as naproxen, flurbiprofen, fenoprofen, ketoprofen and ibuprofen.

The inorganic acid for making strontium salts may be selected from the group consisting of boric acid, bromous acid, chloric acid, diphosphoric acid, disulfuric acid, dithionic acid, dithionous acid, fulminic acid, hydrazoic acid, hydrobromic acid, hydrofluoric acid, hydroiodic acid, hydrogen sulfide, hypophosphoric acid, hypophosphorous acid, iodic acid, iodous acid, metaboric acid, metaphosphoric acid, metaphosphorous acid, metasilicic acid, nitrous acid, orthophosphoric acid, orthophosphorous acid, orthosilicic acid, phosphoric acid, phosphinic acid, phosphonic acid, pyrophosphorous acid, selenic acid, sulfonic acid, thiocyanic acid and thiosulfuric acid.

The organic acid may be selected from the group consisting of C₂H₅COOH, C₃H₇COOH, C₄H₉COOH, (COOH)₂, CH₂(COOH)₂, C₂H₄(COOH)₂, C₃H₆(COOH)₂, C₄H₈(COOH)₂, C₅H₁₀(COOH)₂, 2,3,5,6-tetrabromobenzoic acid, 2,3,5,6-tetrachlorobenzoic acid, 2,3,6-tribromobenzoic acid, 2,3,6-trichlorobenzoic acid, 2,4-dichlorobenzoic acid, 2,4-dihydroxybenzoic acid, 2,6-dinitrobenzoic acid, 3,4-dimethoxybenzoic acid, abietic acid, acetoacetic acid, acetonedicarboxylic acid, aconitic acid, acrylic acid, adipic acid, ascorbic acid, aspartic acid (L and D forms), anthranilic acid, arachidic acid, azelaic acid, behenic acid, benzenesulfonic acid, beta-hydroxybutyric acid, benzilic acid, benzoic acid, brassidic acid, carbonic acid, camphoric acid, capric acid, cholic acid, chloroacrylic acid, cinnamic acid, citrric acid, citraconic acid, crotonic acid, cyclopentane-1,2-dicarboxylic acid, cyclopentanecarboxylic acid, cystathionine, decanoic acid, erucic acid, ethanesulfonic acid, ethylenediaminetetraacetic acid, folic acid, formic acid, fulvic acid, fumaric acid, gallic acid, glutaconic acid, gluconic acid, glutamic acid (L an D), glutaric acid, gulonic acid, heptanoic acid, hexanoic acid, humic acid, hydroxystearic acid, ibuprofenic acid, isophthalic acid, itaconic acid, lactic acid, lanthionine, lauric acid (dodecanoic acid), levulinic acid, linoleic acid (cis,cis-9,12-octadecadienoic acid), malic acid, m-chlorobenzoic acid, malic acid, maleic acid, malonic acid, melissic acid, mesaconic acid, methacrylic acid, methanesulfonic acid, monochloroacetic acid, myristic acid, (tetradecanoic acid), nonanoic acid, norvaline, octanoic acid, oleic acid (cis-9-octadecenoic acid), ornithine, oxaloacetic acid, oxalic acid, palmitic acid (hexadecanoic acid), p-aminobenzoic acid, p-chlorobenzoic acid, petroselic acid, phenylacetic acid, p-hydroxybenzoic acid, pimelic acid, propiolic acid, phthalic acid, propionic acid, p-tert-butylbenzoic acid, p-toluenesulfonic acid, pyruvic acid, ranelic acid, sarcosine, salicylic acid, sebacic acid, serine, sorbic acid, stearic acid (octadecanoic acid), suberic acid, succinic acid, tartaric acid, terephthalic acid, tetrolic acid, L-threonic acid, thyronine, tricarballylic acid, trichloroacetic acid, trifluoroacetic acid, trimellitic acid, trimesic acid, tyrosine, ulmic acid, valeric acid, vanilic acid and cylohexanecarboxylic acid.

In a particular embodiment of the invention, the acid may have a specific pharmacologic action such as a non-steroidal anti inflammatory drug (NSAID). Examples of relevant NSAIDs are enolic acis such as piroxicam and meloxicam, heteroaryl acetic acids such as diclofenac, tolmetin, ketorolac and zomepirac; Indole and indene acetic acids such as indomethacin, mefenamic acid, sulindac and etodolac; propionic acids including naproxen, flurbiprofen, fenoprofen, oxaprozin, carprofen, ketoprofen and ibuprofen; fenamates including mefenamic acid, meclofenamate and flufenamic acid; pyrazolones including phenylbutazone, salicylates including acetyl salicylate (aspirin), salicylate, salsalate, difunisal, olsalazine, fendosal, sulfasalazine and thiosalicylate as well as pharmacologically active derivatives of any of the molecules.

In another embodiment of the invention, the acid may also be an inhibitor of the cyclooxygenase 2 enzyme (COX-2 inhibitor) with an inhibition constant below Ki 10 μm such as lumiracoxib (Prexige), (1,1-dimethylheptyl)-6a,7,10,10a-tetrahydro-1-hydroxy-6,6-dimethyl-6H-dibenzo[b,d]pyran carboxylic acid (CT-3); 2(5H)-Furanone, 5,5-dimethyl (1-methylethoxy) [4(methylsulfonyl)phenyl]-(DFP); flurbiprofene, Carprofen; (Acetyloxy)-benzoic acid, and licofelone [2,2-dimethyl-6-(4-chlorophenyl)-7-phenyl-2,3,dihydro-1H-pyrrolizine-5-yl]-acetic acid as well as pharmacologically active derivatives of any of the molecules.

In another embodiment of the invention, the acid may an inhibitor of inducible NOS (iNOS) such as amino-guanidine, N^{G}-Nitro-L-arginine, N^{G}-Monomethyl-L-arginine, N⁶-(1-Iminoethyl)-L-lysine, N^{G}-Nitro-L-arginine, S-Methyl-L-thiocitrulline, N^{G}-Monomethyl-L-arginine acetate and N^{G}-Monomethyl-L-arginine acetate, 2-Iminopiperidine as well as pharmacologically active derivatives of any of the molecules.

In another embodiment of the invention, the acid may be a Cyclooxygenase (COX)-inhibiting nitric oxide donators (ClNOD) such as AZD3582, AZD4717 and HCT3012, as well as pharmacologically active derivatives of any of the molecules.

However, the present invention is not limited to the above-mentioned specific examples of suitable salts, but merely to the general applicability of water-soluble salts of strontium. Some of the known strontium salts (e.g. strontium chloride and strontium hydroxide) have a very high water-solubility. Irrespective of their water-solubility such strontium salts may be used in the combination treatment of the invention. However, in a specific embodiment of the invention the water-solubility of the strontium salt is at the most about 200 g/l such as, e.g. at the most about 150 g/l, at the most about 100 g/l, at the most about 75 g/l, at the most about 50 g/l, at the most about 25 g/l, at the most about 10 g/l, at the most about 5 g/l, at the most about 2.5 g/l, or at the most about 1 g/l at room temperature (20-25° C.).

In those cases where e.g. a strontium salt having a water-solubility of at the most about 1 g/l (e.g. strontium citrate, strontium carbonate, strontium ranelate, strontium oxalate or strontium hydrogen phosphate), the present inventors have shown that it is possible to delay the appearance of the peak concentration, i.e. the active substance itself may contribute to a delayed release of the strontium ion. This may provide a therapeutic benefit when administered in combination with another pharmaceutical substance as defined in the present invention. Such delayed release properties will be especially relevant in combination treatments according to the present invention, where one or more of the active pharmaceutical substances has the propensity to induce gastrointestinal (GI) damage such as epigastric/abdominal pain, nausea, vomiting, diarrhea, dyspepsia, bloating, flatulence, anorexia, mucosal erosions and/or inflammation (esophagitis, gastritis, duodenitis, enteritis), gastrointestinal hemorrhage including hematemesis, melena and hematochezia, (peptic) ulcerations and GI strictures. Increased susceptibility/risk for GI side effects is particularly associated with the intake of NSAIDs, COX-2 inhibitors, COX-3 inhibitors, Cyclooxygenase (COX)-inhibiting nitric oxide donators (ClNOD). Especially if the treatment is given in the evening, it can be advantageous to have a sustained release of the active strontium ion, as this will allow the strontium to exert its GI-protective effect throughout the night. Thus a sustained release of strontium ions throughout the night must be expected to provide the greatest physiological effect.

Moreover, in a specific embodiment of the invention, the strontium salt for use according to the invention may be water soluble, having a water solubility of at least 1 g/l, such as, e.g., at least 5 g/l, at least 10 g/l, at least 20 g/l, at least 30 g/l, at least 40 g/l, at least 50 g/l, at least 60 g/l, at least 70 g/l, at least 80 g/l, at least 90 g/l or at least 100 g/1 measured at room temperature, i.e. a temperature of 20-25° C. A more water soluble organic strontium salt comprising an anion with one or more carboxyl-groups may provide significant physiological benefits for a medical use according to the invention.

We have found that such salts, due to the intrinsic alkaline properties of ionic strontium elevates pH when solubilised in aqueous media, such as the gastric juice of the stomach, thereby providing a maximal GI-protective effect. Thus, when administered in combination with one or more further palliative agents according to the present invention, selected from the pharmaceutical drug classes comprising NSAIDs, COX-2 inhibitors, iNOS inhibitors, Neuroleptic agents and Cyclooxygenase (COX)-inhibiting nitric oxide donators (ClNOD) which are known to be associated with significant gastrointestinal (GI) adverse events, the strontium salt will have a beneficial effect and serve to prevent or reduce occurrence of GI adverse events, which is of significant concern in long term palliative treatment for management of conditions of chronic pain.

In one embodiment, the present invention can be carried out by combining in one pharmaceutical formulation a strontium compound in combination with one or more palliative agents according to the present invention, selected from the pharmaceutical drug classes comprising NSAIDs, COX-2 inhibitors, COX-3 inhibitors, iNOS inhibitors, PAR2 receptor antagonists, Neuroleptic agents, Opioids, N-acetylcholine receptor agonists, glycine antagonists, vanilloid receptor antagonists, neurokinin antagonists calcitonine gene-related peptide antagonists and Cyclooxygenase (COX)-inhibiting nitric oxide donators (ClNOD). Such combinations may be administered separately to a subject in need thereof, or they may be given in combination formulated in the same pharmaceutical dosage unit. Pharmaceutical compositions comprising an effective amount of a strontium containing compound according to the invention and another palliative agent according to the invention may conveniently be formulated with suitable carrier or diluent. Such compositions are preferably in the form of an oral dosage unit or parenteral dosage unit.

Accordingly, in a preferred embodiment, the invention relates to a pharmaceutical composition comprising a) a strontium-containing compound and b) one or more further palliative agents selected from the therapeutic drug classes comprising NSAIDs, COX-2 inhibitors, COX-3 inhibitors, iNOS inhibitors, PAR2 receptor antagonists, Neuroleptic agents, Opioids, N-acetylcholine receptor agonists, glycine antagonists, vanilloid receptor antagonists, neurokinin antagonists calcitonine gene-related peptide antagonists and Cyclooxygenase (COX)-inhibiting nitric oxide donators (ClNOD) formulated with physiologically acceptable excipients.

The physiologically acceptable excipients may be a therapeutically inert substance or carrier.

The carrier may take a wide variety of forms depending on the desired dosage form and administration route.

The pharmaceutically acceptable excipients may also be e.g. fillers, binders, disintegrants, diluents, glidants, solvents, emulsifying agents, suspending agents, stabilizers, enhancers, flavors, colors, pH adjusting agents, retarding agents, wetting agents, surface active agents, preservatives, antioxidants etc. Details can be found in pharmaceutical handbooks such as, e.g., Remington's Pharmaceutical Science or Pharmaceutical Excipient Handbook.

The compounds with which the invention is concerned may also be prepared for administration by any route consistent with their pharmacokinetic properties. Especially an oral administration of one or more pharmaceutical compounds according to the invention is relevant, as this is a likely administration route where GI side effects are encountered. The orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, carboxy-methyl cellulose, cyclo-dextrin, dextrose, corn-starch, calcium phosphate, sorbitol or glycine; tabletting lubricant, for example magnesium stearate, talc, polyethylene glycol or silica; disintegrants for example potato starch, corn starch, polyvinyl-pyrolidone, or acceptable wetting agents and glidants such as sodium lauryl sulphate or magnesium stearate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan mono-oleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or coloring agents.

The solid composition may be in the form of tablets such as, e.g. conventional tablets, effervescent tablets, coated tablets, melt tablets or sublingual tablets, pellets, powders, granules, granulates, particulate material, solid dispersions or solid solutions.

In one embodiment of the invention, the pharmaceutical composition may be in the form of a tablet. The tablet may be coated with a coating that enables release of at least part of the salt in the proximal part of the small intestine, such as e.g. the duodenum and/or the proximal jejunum such as at least 50% w/w, at least 60% w/w, at least 65% w/w, at least 70% w/w, at least 80% w/w or at least 90% w/w of the total amount of the salt contained in the tablet.

The tablet may have a shape that makes it easy and convenient for a patient to swallow. The tablet may thus e.g. have a rounded or a rod-like shape without any sharp edges. Furthermore, the tablet may be designed to be divided in two or more parts.

### DEFINITIONS

### NSAIDs

For the scope of this invention the class of compounds categorized as non-steroidal antiinflammatory agents (hereinafter NSAID's) comprise molecules such as piroxicam, diclofenac, propionic acids such as naproxen, flurbiprofen, fenoprofen, ketoprofen and ibuprofen, fenamates such as mefenamic acid, indomethacin, sulindac, meloxicam, apazone, pyrazolones such as phenylbutazone, salicylates such as aspirin, COX-2 inhibitors such as celecoxib (tradename CELEBREX® by G. D. Searle & Co., Skokie, Ill.), valdecoxib (tradename BEXTRA® by Pharmacia & Upjohn Company, North Peapack, N.J.), etoricoxib (tradename ARCOXIA® by Merck & Co., Inc., Whitehouse Station, N.J.), lumiracoxib (tradename PREXIGE® by Novartis AG, Basel, Switzerland), parecoxib, and rofecoxib (tradename VIOXX® by Merck & Co., Inc., Whitehouse Station, N.J.), deracoxib (tradename DERAMAXX® by Novartis AG, Basel, Switzerland).

Selective COX-2 inhibitors

COX-2 inhibitors may be considered a subgroup of the NSAID class of analgesic/anti-inflammatory agents optimized to reduce side effects. For the purposes of this invention, a selective inhibitor of COX-2, is defined as a compound that shows a preferential inhibition of the COX-2 isoenzyme compared to the COX-1 isoenzyme, such as at least a 5 fold lower IC₅₀ for the COX-2 enzyme compared IC₅₀ for COX-1, or even more preferred a 10 fold lower IC₅₀ for COX-2. In a preferred embodiment of the present invention the COX-2 selective inhibitors do not display any inhibition of the 5-Lipoxygenase (5-LOX) enzyme at a concentration of 10 μM. As specific examples of COX-2 inhibitors reference is made to the compounds disclosed in the prior art reference as COX-2 inhibitors. This group of compounds includes the following substances, or a pharmaceutically acceptable salt thereof, selected from the group comprising: LAS-34475; UR-8880; ABT-963; Valdecoxib; BMS-347070; Celecoxib; Tilacoxib; (1,1-dimethylheptyl)-6a,7,10,10a-tetrahydro-1-hydroxy-6,6-dimethyl-6H-dibenzo-[b,d]-pyran carboxylic acid (“CT-3”); CV-247; 2(5H)-Furanone, 5,5-dimethyl (1-methylethoxy) [4(methylsulfonyl)phenyl]-(“DFP”); CS-502 (CAS Reg. nr. 176429-82-6); Carprofen (trade name RIMADYLO® by Pfizer, Inc., New York, N.Y.); Deracoxib (tradename DERAM® by Novartis AG, Basel, Switzerland); Etoricoxib (tradename ARCOXIA® by MERCK & CO., Inc., Whitehouse Station, N.J.); GW-406381; Tiracoxib; Meloxicam; Nimesulide; 2-(Acetyloxy)benzoic acid, 3-[(nitrooxy)methyl]phenyl ester (“NCX4016”); Lumiracoxib (tradename PREXIGEO by Novartis AG, Basel, Switzerland); Parecoxib (trade name application pending for DYNASTAT® by G. D. Searle & Co., Skokie, Ill.); P54 (CAS Reg. No. 130996 0); Rofecoxib (tradename VIOXX® by MERCK & CO., Inc., Whitehouse Station, N.J.); 2,6-Bis(1,1-dimethylethyl) [(E)-(2-ethyl-1,1-dioxo isothiazolidinylidene)methyl]phenol (“S-2474”); 5(R)-Thio sulfonamide-3(2H)-benzofuranone (“SVT-2016”); and N-[3-(Formyl-amino) oxo phenoxy-4H benzopyran yl]methanesulfonamide (“T-614”); or a pharmaceutically acceptable salt thereof. The terra “celecoxib” means the compound named 4-(5-(4-methylphenyl) 3-(trifluoromethyl)-1H-pyrazol-t-yl)-benzenesulfonamide. Celecoxib is a selective COX-2 inhibitor currently approved by the FDA for the treatment of osteoarthritis, rheumatoid arthritis, and Polyposis-familial adenomatus. Celecoxib is marketed under the tradename “CELEBREX®”. Celecoxib is currently in clinical trials for the treatment of bladder cancer, chemopreventative-lung cancer, and post-operative pain, and is registered for the treatment of dysmenorrhea.

The term “valdecoxib” means the compound named 4-(5-methyl phenyl-4-isoxazolyl)-benzenesulfonamide, which is described in U.S. Pat. Nos. 5,633,272; 5,859,257; and 5,985,902, which are hereby incorporated by reference herein. Valdecoxib has been approved by the FDA for treating osteoarthritis, rheumatoid arthritis, dysmenorrhea, and general pain, and is marketed under the tradename “BEXTRA®”.

In addition to the specific examples of COX-2 selective compounds listed above, a great number of selective COX-2 inhibitors are disclosed in the prior art literature and may be used in a pharmaceutical composition according to the present invention. Examples of COX-2 inhibitors are disclosed in, for example, U.S. Pat. Nos. 5,681,842; 5.750,558; 5,756,531; 5,776,984 and in WO 97/41100, WO 98/39330, WO 99/10331, WO 99/10332 and WO 00/24719 assigned to Abbott Laboratories; and in WO 98/50075, WO 00/29022 and WO 00/29023 assigned to Algos Pharmaceutical Corporation; and in WO 99/15205 assigned to Ahnirall Prodesfarma S.A.; and in U.S. Pat. No. 5,980,905 assigned to AMBI Inc.; and in U.S. Pat. No. 5,945,538 assigned to American Cyanamid Company; and in U.S. Pat. Nos. 5,776,967, 5,824,699; 5,830,911 and in WO 98/04527 and WO 98/21195 assigned to American Home Products Corporation; and in WO 98/22442 assigned to Angelini Richerche S.P.A. Societa Consortile; and in U.S. Pat. No. 6,046,191 and in WO 99/18960 and WO 00/00200 assigned to Astra Pharmaceuticals Ltd.; and in U.S. Pat. No. 5,905,089; assigned to Board of Supervisors of Louisiana State University; and in U.S. Pat. Nos. 5,620,999; 5,633,272; 5,643,933, 5,668,161; 5,686,470; 5,696,431; 5,719,163; 5,753,6881; 5,756,530; 5,760,068; 5,859,2571; 5,908,852; 5,935,990; 5,972,986; 5,985,902; 5,990,148; 6,025,353; 6,028,072; 6,136,839 and in WO 94/15932; WO 94/27980; WO 95/11883; WO 95/15315; WO 95/15316; WO 95/15317; WO 95/15318, WO 95/21817; WO 95/30652; WO 95/30656; WO 96/03392; WO 96/03385; WO 96/03387; WO 96/03388; WO 96/09293; WO 96/09304; WO 96/16934; WO 96/25405; WO 96/24584; WO 96/24585; WO 96/36617; WO 96/384181; WO 96/38442; WO 96/41626; WO 96/41645; WO 97/11704; WO 97/27181; WO 97/29776; WO 97/38986; WO 98/06708; WO 98/43649; WO 98/47509; WO 98/47890, WO 98/52937; WO 99/22720; WO 00/23433; WO 00/37107; WO 00/38730; WO 00/38786 and WO 00/53149 assigned to G.D. Searle & Co.; and in WO 96/31509; WO 99/12930; WO 00/26216 and WO 00/52008 assigned to Glaxo Group Limited; and in EP 1 006 114 Al and in WO 98/46594 assigned to Grelan Pharmaceutical Co. Ltd.; and in WO 97/34882 assigned to Gruppo Farmaceutico Almirall- and in WO 97/03953 assigned to Hafslund Nycomed Pharma AG; and in WO 98/32732 assigned to Hoffmann-La Roche AG; and in U.S. Pat. Nos. 5,945,539; 5,994,381; 6,002,014 and in WO 96/19462; WO 96/19463 and in EP 0 745 596 Al assigned to Japan Tobacco, Inc.; and in U.S. Pat. Nos. 5,686,460; 5,807,873 and in WO 97/37984; WO 98/05639; WO 98/11080 and WO 99/21585 assigned to Laboratories USPA; and in WO 99/62884 assigned to Laboratories Del Dr. Esteve, S.A.; and in WO 00/08024 assigned to Laboratorios S.A.L.V.A.T., S.A.; and in U.S. Pat. Nos. 5,585,504; 5,840,924; 5,883,267; 5,925,631; 6,001,843; 6,080,876 and in WO 97/44027; WO 97/44028; WO 97/45420; WO 98/03484; WO 98/41511; WO 98/41516; WO 98/43966; WO 99/14194; WO 99/14195; WO 99/23087, WO 99/41224 and WO 00/68215 assigned to Merck Frosst Canada & Co., and in WO 99/59635 assigned to Merck Sharp & Dohme Limited; and in U.S. Pat. No. 5,380,738 assigned to Monsanto Company; and in WO 00/01380 assigned to A. Nattermann & Co.; and in WO 99/61016 assigned to Nippon Shinyaku Co. Ltd.; and in WO 99/33796 assigned to Nissin Food Products Co. Ltd.; and in WO 99/11605 assigned to Novartis AG; and in WO 98/33769 assigned to Nycomed Austria GMBH; and in U.S. Pat. Nos. 6,077,869 and 6,083,969 and in WO 00/51685 assigned to Ortho-McNeil Pharmaceutical, Inc.; and in U.S. Pat. No. 5,783,597 assigned to Ortho Pharmaceutical Corporation; and in WO 98/07714 assigned to Oxis International Inc.; and in WO 00/10993 assigned to Pacific Corporation; and in EP 0 937 722 A1 and in WO 98/50033; WO 99/05104; WO 99/35130 and WO 99/64415 assigned to Pfizer Inc.; and in WO 00/48583 assigned to Pozen Inc.; and in U.S. Pat. No. 5,908,858 assigned to Sankyo Company Limited; and in WO 97/25045 assigned to SmithKline Beecham Corporation; and in U.S. Pat. No. 5,399,357 assigned to Takeda Chemical Industries, Ltd.; and in WO 99/20589 assigned to The University of Sydney; and in U.S. Pat. No. 5,475,021 and WO 00/40087 assigned to Vanderbilt University; and in WO 99/59634 assigned to Wakamoto Pharmaceutical Co. Ltd., the disclosures of each of which are incorporated by reference herein in their entirety.

### Neuroleptics

Neuroleptics (antipsychotics) is a common term for a diverse group of pharmaceutical substances with the ability to inhibit dopamine nerve transmission in the frontal lobes and in the emotion-regulating limbic system of the brain. Among the most commonly employed neuroleptic agents are compounds comprising a phenothiazine group, such as Fluphenazine (common trade names Permitil and Prolixin, typically administered in doses of 2.5-10 mg/day), prochlorperazine (Compazine, typically administered in doses of 5-10 mg/day), Trifluoperazine (Stelazine, typically administered in doses of 1-10 mg/day), Perphenazine (Trilafon and Etrafon, typically administered in doses of 2-16 mg/day), Chlorpromazine (Thorazine, typically administered in doses of 10-200 mg/day), Thioridazine (Mellaril, typically administered in doses of 10-200 mg/day), mesoridazine besylate (Serentil, typically administered in doses of 25-100 mg/day); compounds comprising a thiozhanthene moiety such as thiothixine (Navane, typically administered in doses of 1-20 mg/day); compounds comprising a butyrophenone group such as haloperidol (Haldol, typically administered in doses of 0.5-20 mg/day); compounds belonging to the thieno-benzodiazepine class such as olanzapine (Zyprexa, typically administered in doses of 2.5-20 mg/day) and a number of other heterocyclic and/or aliphatic compounds such as molindone (Moban, typically administered in doses of 5-100 mg/day), loxapine (Loxitane, typically administered in doses of 5-50 mg/day), pimozide (Orap, typically administered in doses of 2 mg/day), clozapine (Clozaril, typically administered in doses of 25-100 mg/day), risperidone (Risperdal, typically administered in doses of 1-4 mg/day), quetiapine (Seroquel, typically administered in doses of 25-200 mg/day), Chlorprothixene (Taractan, typically administered in doses of 10-100 mg/day), droperidol (Inapsine, typically administered in doses of 5-100 mg/day), Promethazine (Phenergan), Amitriptyline (Triavil), ziprasidone (Geodon, typically administered in doses of 20-80 mg/day), metoclopramide (Reglan, typically administered in doses of 5-10 mg/day).

### ClNODs

A new class of analgesic agents with the ability to inhibit the COX enzymes has recently been described. This is the so-called Cyclooxygenase (COX)-inhibiting nitric oxide donators (ClNOD). This class of compound is being developed for the treatment of acute and chronic nociceptive pain, such as post-operative and arthritic pain. AZD3582 is an entirely new chemical entity which provides balanced inhibition of COX enzymes while also donating nitric oxide at sites of inflammation. This mode of action may reduce inflammation, as nitric oxide is known to exert a relaxing effect on endothelial cells. Donation of nitric oxide may also have a protective effect on the gastrointestinal tract and other organs. Damage to the gastrointestinal tract is a known side effect of conventional NSAID use and is believed to be associated with inhibition of COX-1. Other members of this emerging pharmaceutical class are the compounds AZD4717 and HCT3012.

### Opioids

For the purpose of this invention the term ‘opioid’s' may be considered to comprise both naturally occurring compounds including endorphins, nociceptin, endomorphins, and synthetically manufactured compounds with the common property of being able to bind opioid receptors in the central nervous system (CNS) as well as in the periphery, thereby providing a substantial palliative effect. Any compound with the ability to bind an opioid receptor with an affinity constant below 10 mM, preferable below 1 mM, more preferably below 0.1 mM or even more preferably below 10 μM can be used to carry out the present invention, but in a preferred embodiment of the invention a selective agonist of the mu-1 receptor is used. Of pharmaceutical relevance for the present invention are also compounds acting as specific canabinoid receptor antagonists. Examples of opioids include Heroin, fentanyl, morphine, oxycodone, hydrocodone, methadone, buprenorphine, pentazocine, butorphanol, dezocine, nalbuphine, Meperidine, normeperidine, hydromorphone, codeine, levorphanol and tramadol, BW373U86, CP 55,940 and SNC-121, and active metabolites thereof.

Inhibitors of Inducible Nitric Oxide Synthetase (iNOS)

Nitric oxide is synthesized by the action of nitric oxide synthetases or NOS enzymes on the amino acid L-arginine. Endothelial NOS (eNOS) of the blood vessels and neuronal NOS (nNOS) in neurons continuously produce low levels of NO that is used in blood pressure regulation and neurotransmission, respectively. The inducible NOS (iNOS) gene is expressed as a result of stimulation by inflammatory cytokines and is an important component in the body's immune defense repertoire. All forms of NOS catalize the same chemical reaction where nitric oxide and L-citrulline is formed by hydrolysis and reduction of L-arginine via N^{G}-hydroxy-L-arginine (L-NOHA) using NADPH, FMN, FAD and tetrahydrobiopterin as co-enzymes. During inflammation, the iNOS enzyme is expressed in many tissues and produces NO at levels 1,000-times greater than nNOS or eNOS. Excessive NO production from iNOS is a major contributor to the pathology of many diseases, and thus a number of specific reversible as well as irreversible inhibitors of iNOS have been introduced in clinical practice for the management of pain in chronic as well as acute disorders.

The first generation of iNOS inhibitors comprise amino-guanidine, N^{G}-Nitro-L-arginine and N^{G}-Monomethyl-L-arginine which can be considered as analogues of the natural amino acid L-arginine which has a nitro group on the N^{G} of the guanidino moiety. N⁵-(1-Iminoethyl)-L-ornithine is an L-ornithine analog which has an iminoethyl group instead of an amine group and it is an irreversible inhibitor of iNOS in phagocytic cells, and is a reversible inhibitor in endothelial cells. N⁶-(1-Iminoethyl)-L-lysine is an L-lysine analog which as an iminoethyl group instead of an amine group. N⁶-(1-Iminoethyl)-L-lysine is known as an irreversible NOS inhibitor. L-Thiocitrulline, is a selective inhibitor for endothelial and neuronal NOS, which inhibits the NO production by decreasing the reducing potential of heme. N^{G}-Nitro-L-arginine is a selective inhibitor of endothelial and neuronal NOS. S-Methyl-L-thiocitrulline, an analog of L-citrulline, is more potent than an L-arginine analog such as N^{G}-Monomethyl-L-arginine acetate. S-Methyl-L-thiocitrulline inhibits the oxidation of L-arginine and L-arginine-dependent oxidation of NADPH by neuronal NOS from the human brain. Diphenyleneiodonium chloride has been shown to inhibit NOS in cultured mouse macrophages. It is known that isothiourea derivatives, such as S-Methylisothiourea, S-Ethylisothiourea, S-Isopropylisothiourea, and S-(2-Aminoethyl)-isothiourea, inhibit iNOS highly selectively. EC50 of these derivatives are reported as 6 mM, 2 mM, 2 mM, 3 mM, respectively against macrophage cells J774.2 treated with bacterial endotoxin. They are 8 to 24 times more potent than N^{G}-Monomethyl-L-arginine acetate. Also it has been reported that these isothiourea derivatives are 2 to 19 times more selective to human iNOS than that of mice. 2-Iminopiperidine is another selective and potent inhibitor to human iNOS. 2-Iminopiperidine inhibits human iNOS at lower concentration than other inhibitors, and further, it inhibits nNOS strongly. 2,4-Diamino-6-hydroxy-pyrimidine inhibits the activity of GTP cyclohydrolase (GTPCH), which converts GTP to tetrahydrobiopterin (BH4). BH4 is a co-factor of iNOS, and is produced by the enzymatic reaction of GTPCH, sepiapterin reductase, or aldose reductase.

Currently there is a number of specific iNOS inhibitors in clinical developments such as 5-chloro-1,3-dihydro-2H-benzimidazol-2-one (FR038251), 1,3(2H,4H)-isoquinoline-dione (FR038470) and 5-chloro-2,4(1H,3H)-quinazolonedione (FR191863), which show inhibition of inducible nitric oxide synthase (iNOS). These families of compounds have shown great potential for palliative interventions in relevant preclinical studies as well as in clinical investigations.

### Other Palliative Treatments

A number of physiological drug targets have been implicated in the regulation of pain either systemically and/or locally, and a number of pharmaceutical substances are currently being developed to target these molecules, receptors and enzymes. COX-3 inhibitors, PAR2 receptor antagonists, N-acetylcholine receptor agonists, glycine antagonists, vanilloid receptor antagonists, neurokinin antagonists, NMDA receptor antagonists and calcitonin gene-related peptide antagonists.

COX-3 inhibitors represent an emerging new class of analgesic agents. This COX isoform can be considered a special variant of COX-1. COX-3, as well as two smaller COX-1-derived proteins (partial COX-1 or PCOX-1 proteins) is made from the COX-1 gene but retain intron 1 in their mRNAs. PCOX-1 proteins additionally contain an in-frame deletion of exons 5-8 of the COX-1 mRNA. COX-3 and PCOX mRNAs are expressed in canine cerebral cortex and in lesser amounts in other tissues analyzed. In human, COX-3 mRNA is expressed as an approximately 5.2-kb transcript and is most abundant in cerebral cortex and heart. Acetaminophen and other analgesic/antipyretic drugs have been shown to inhibit COX-3, and now development of new specific inhibitors of this enzyme is underway in order to determine their potential in palliative treatments.

Another group of pharmaceutical agents of relevance for the present invention is comprised by antagonists of neurokinin-1 (NK(1)) receptors, through which substance P acts. This class of molecules have been proposed to belong to a new class of antidepressants with a unique mode of action, but as is known that substance P is a mediator of pain signaling, especially in soft tissues, NK(1) receptor antagonist may also hold a potential as palliative agents. Several non-peptidic NK(1) antagonist has been described, e.g. CP-96,345. Also selective neurokinin-2 (NK(2)) receptor antagonist, such as SR48968 may be useful in a combination therapy according to the present invention.

Another pharmaceutical class of compounds of relevance for the present invention is represented by vanilloid receptor antagonists. Such compounds may be considered a sub-class of the Opioid group of compounds. Vanilloid receptors are predominantly expressed on C and A fibers projecting to the dorsal horn of the spinal cord, as well as in trigeminal ganglion neurons projecting to the spinal nucleus of the trigeminal tract. Vanilloids, such as capsaicin, elicit a biphasic action on sensory neurons characterized by an initial exctitatory phase (pain and/or inflammation) followed by desensitization. Thus specific antagonists of this class of CNS receptors can be used in the treatment of pain. A number of vanilloid receptor antagonists are known from the prior art literature such as Arvanil, Isovelleral, Olvanil, 5′-Iodoresiniferatoxin, Phorbol 12,13-didecanoate 20-homovanillate, Phorbol 12,13-dinonanoate 20-homovanillate, SB-366791, Scutigeral and Anti-Vanilloid Receptor-Like Protein 1, all of which may hold a potential as palliative agents.

The amino acid glycine is one of the major inhibitory neurotransmitters in the mammalian CNS, predominantly active in the spinal cord and brain stem. Although not primarily involved in mediation of pain sensation glycine can be involved in some forms of sensatory signaling and accordingly may comprise a pharmaceutical class of relevance for the present invention. In conjunction with the pharmaceutical role of glycine antagonists and/or agonist it is also relevant to include the N-methyl-D-aspartate (NMDA) receptor as a potential drug target of relevance for the present invention. NMDA Also acts as a modulator of excitatory amino acid transmission. A number of partial glycine agonists (such as e.g. R(+)-3-Amino-1-hydroxypyrrolidin-2-one and 1-Amino-cyclobutanecarboxylic acid) and full glycine antagonists (such as ACEA-1328 and various derivatives of tetrahydroquinolines) are known in the literature. Furthermore, a great number of NMDA receptor antagonists are known, such as MK801, dextromethorphan (DM), ketamine, phencyclidine (PCP), LY274614, NPC17742, LY235959 [(1S)-1-[[(7-Bromo-1,2,3,4-tetrahydro-2,3-dioxo-5-quinoxalinyl)methyl]amino]ethyl]-phosphonic acid; 7-Chloro-4-hydroxyquinoline-2-carboxylic acid; 5,7-Dichloro-4-hydroxy-quinoline-2-carboxylic acid; trans-2-Carboxy-5,7-dichloro-4-phenylaminocarbonylamino-1,2,3,4-tetrahydro-quinoline and 7-Chloro-4-hydroxy-3-(3-phenoxy)phenyl-2(H)-quinolinone),

An additional class of compounds of relevance for the present invention is represented by 6-(5-carboxy methyl-hexyloxy)-2,2-dimethyl-hexanoic acid, calcium salt, and analogues as disclosed in patents WO 04/017952 and WO 03/003664, which are hereby include by reference in their entirety. The method of action of this class of anti-rheumatic and/or anti-inflammatory agents has not been completely resolved, but it is believed that they act through IL-6 mediated pathways, thereby providing a therapeutic options in all disease states and pathologies in which aberrant regulation of IL-6 has been implicated.

Any one of the substances listed above, selected from the groups comprising NSAIDs, COX-2 inhibitors, COX-3 inhibitors, iNOS inhibitors, PAR2 receptor antagonists, Neuroleptic agents, Opioids, COX-3 inhibitors, PAR2 receptor antagonists, N-acetylcholine receptor agonists, glycine antagonists, vanilloid receptor antagonists, neurokinin antagonists, NMDA receptor antagonists and calcitonin gene-related peptide antagonists or other palliative agents or any combinations thereof may be used to carry out the present invention.

Furthermore, it follows that a person skilled in the art may devise derivatives of any one of the organic molecules listed above such as, but not limited to, esters, salts, alkylated forms, forms modified by attachment of side-groups selected from the group comprising halogen, alkyl, halogenoalkyl, alkoxy, aryloxy, halogenalkoxy, alkylthio, lower alkylene radical, hydroxyl, nitro, alkylsulfinyl, alkylsulfonyl, sulfamoyl, N-alkylsulfamoyl; aza-, oxa- or thia-lower alkylene radicals, such as 3- or 4-aza-lower alkylene that is unsubstituted or N-substituted by lower alkyl, hydroxy-lower alkyl, lower alkoxy-lower alkyl or by lower alkanoyl, 3- or 4-oxa-lower alkylene or optionally S-oxidised 3- or 4-thia-lower alkylene or another aliphatic group such as a phenyl, thiophenyl, thiophene, fumarate, furan, pyrrole, pyridine, piperidine, imidazole, quinoline, isoquinoline or carbazole group in either unsubstituted form or substituted with one or more lower alkyl or hydroxyl-alky, or amino alkyl groups having from 1 to 7 carbon atoms.

The invention is further illustrated by the following non-limiting examples. Specific aspects and embodiments appear from the appended claims.

### EXAMPLES

### Example 1

### Pharmaceutical Composition Containing Naproxen and a Strontium Compound

Tablet formulation

| Ingredient | Amount (mg)/tablet |
|---|---|
| Naproxen | 250 mg |
| Strontium malonate | 210 mg |
| Lactose Ph.Eur. | 100 mg |
| Corn starch Ph.Eur. (for mixing) | 15 mg |
| Corn starch Ph.Eur. (for paste) | 15 mg |
| Magnesium Stearate Ph.Eur. (1%) | 10 mg |
| Total | 500 mg |

Naproxen and strontium malonate, lactose and cornstarch (for mixing) are blended to uniformity. The cornstarch for paste is suspended in 200 ml of water and heated with stirring to form a paste. The paste is used to granulate the mixed powders (wet granulation). The wet granules are passed through a number 8 hand screen and dried at 80° C. After drying, the granules are lubricated with 1% magnesium stearate and pressed into a tablet. Such tablets can be administered to a human subject in need thereof, such as an OA or RA patient, from one to two times daily

### Example 2

### Pharmaceutical Composition Containing Celecoxib and a Strontium Compound

Tablet formulation

| Ingredient | Amount (mg)/tablet |
|---|---|
| Celecoxib | 200 mg |
| Strontium malonate | 200 mg |
| Lactose Ph.Eur. | 100 mg |
| Corn starch Ph.Eur. (for mixing) | 15 mg |
| Corn starch Ph.Eur. (for paste) | 15 mg |
| Magnesium Stearate Ph.Eur. (1%) | 10 mg |
| Total | 540 mg |

The tablets are prepared as described in Example 1.

### Example 3

### Composition Comprising a Combination of a Strontium Containing Compound and 6-(5-carboxy-methyl-hexyloxy)-2,2-dimethyl-hexanoic Acid

Tablet formulation

| Ingredient | Amount (mg) |
|---|---|
| 6-(5-carboxy-methyl-hexyloxy)-2,2-dimethyl- | 20 mg |
| hexanoic acid, strontium salt | |
| strontium malonate | 520 mg |
| Lactose | 20 mg |
| Corn starch (for mixing) | 15 mg |
| Corn starch (for paste) | 15 mg |
| Magnesium Stearate (1%) | 10 mg |
| Total | 600 mg |

6-(5-carboxy methyl-hexyloxy)-2,2-dimethyl-hexanoic acid, strontium salt and strontium malonate, lactose and cornstarch (for mixing) is blended to uniformity. The cornstarch for paste is suspended in 200 ml of water and heated with stirring to form a paste. The paste is used to granulate the mixed powders (wet granulation). The wet granules are passed through a number 8 hand screen and dried at 80° C. After drying, the granules are lubricated with 1% magnesium stearate and pressed into a tablet. Such tablets can be administered to a human subject in need thereof, such as an OA or RA patient, from one to two times daily.

1. A pharmaceutical composition comprising
i) a strontium containing compound and
ii) a second therapeutically and/or prophylactically active substance selected from the group consisting of analgesic agents, anti-inflammatory agents and palliative agents.

2. The pharmaceutical composition according to claim 1, wherein the second therapeutically and/or prophylactically active substance is selected from the group consisting of NSAIDs, COX-2 inhibitors, COX-3 inhibitors, iNOS inhibitors, PAR2 receptor antagonists, neuroleptic agents, opioids, ClNOD, COX-3 inhibitors, PAR2 receptor antagonists, N-acetylcholine receptor agonists, glycine antagonists, vanilloid receptor antagonists, neurokinin antagonists, NMDA receptor antagonists, calcitonin gene-related peptide antagonists and 6-(5-carboxy methyl-hexyloxy)-2,2-dimethyl-hexanoic acid and analogues thereof including active metabolites thereof.

3. The pharmaceutical composition according to claim 1, wherein the strontium-containing compound is selected from the group of organic strontium salts comprising: strontium malonate, strontium succinate, strontium fumarate, strontium ascorbate, strontium aspartate in either L and/or D-form, strontium glutamate in either L- and/or D-form, strontium pyruvate, strontium tartrate, strontium glutarate, strontium maleate, strontium methanesulfonate, strontium benzenesulfonate and strontium ranelate, strontium acetyl salicylate, strontium salicylate, strontium citrate, strontium alendronate, strontium risedronate, strontium chlodronate, strontium ethidronate and strontium L-threonate, strontium ibandronate, strontium ibuprofenate, strontium flubiprofenate, strontium ketoprofenate, strontium phorbol 12,13-didecanoate 20-homovanillate, strontium indomethacinate, strontium carprofenate, strontium naproxenate, strontium acetyloxy-benzoate, strontium 2-Iminopiperidine, strontium methotrexate, strontium salsalate and strontium sulfasalazinate.

4. The pharmaceutical composition according to claim 1, wherein the strontium containing compound is 6-(5-carboxy methyl-hexyloxy)-2,2-dimethyl-hexanoic acid, strontium salt.

5. The pharmaceutical composition according to claim 1, wherein the second therapeutically and/or prophylactically active substance is an NSAID selected from the group consisting of piroxicam, diclofenac, propionic acids, fenamates, paracetamol, indomethacin, sulindac, meloxicam, apazone, pyrazolones, and salicylates.

6. The pharmaceutical composition according to claim 1, wherein the second therapeutically and/or prophylactically active substance is a selective COX-2 inhibitor that has at least a 10 fold higher affinity for the COX-2 enzyme compared to the COX-1 enzyme.

7. The composition according to claim 6 where the COX-2 inhibitor is defined as not having an inhibitory action on the human 5-lipoxygenase (5-LOX) enzyme in a concentration of 10 mM.

8. The pharmaceutical composition according to claim 1, wherein the second therapeutically and/or prophylactically active substance is a COX-2 inhibitor selected from the group consisting of rofecoxib (Vioxx), valdecoxib (Bextra), celecoxib (Celebrex), etoricoxib (Arcoxia), lumiracoxib (Prexige), parecoxib (Dynastat), deracoxib (Deram), tiracoxib, meloxicam, nimesolide, (1,1-dimethylheptyl)-6a,7,10,10a-tetrahydro-1-hydroxy-6,6-dimethyl-6H-dibenzo[b,d]pyran carboxylic acid (CT-3); 2(5H)-Furanone, 5,5-dimethyl (1-methylethoxy) [4(methylsulfonyl)phenyl]-(DFP); Carprofen (RIMADYLO); (Acetyloxy)-benzoic acid, 3-[(nitrooxy)-methyl]phenyl ester (NCX4016); P54 (CAS Reg. No. 1309960) 2,6-Bis(1,1-dimethylethyl) [(E)-(2-ethyl-1,1-dioxo isothiazolidinylidene)-methyl]phenol (S-2474); liclofelone (ML3000); 5(R)-Thio sulfonamide-3(2H)-benzofuranone (SVT-2016) and N-[3-(Formyl-amino) oxo phenoxy-4H benzopyran yl]methanesulfonamide (“T-614”), and pharmaceutically acceptable salts thereof.

9. The pharmaceutical composition according to claim 1, wherein the second therapeutically and/or prophylactically active substance is a Cyclooxygenase (COX)-inhibiting nitric oxide donators (ClNOD) selected from the group consisting of AZD3582, AZD4717 and HCT3012, and therapeutically active derivatives thereof.

10. The pharmaceutical composition according to claim 1, wherein the second therapeutically and/or prophylactically active substance is an inhibitor of inducible NOS (iNOS) selected from the group consisting of amino-guanidine, N^{G}-Nitro-L-arginine, N^{G}-Monomethyl-L-arginine, N⁶-(1-Iminoethyl)-L-lysine, N^{G}-Nitro-L-arginine, S-Methyl-L-thiocitrulline, N^{G}-Monomethyl-L-arginine acetate, diphenyleneiodonium chloride, isothiourea derivatives, N^{G}-Monomethyl-L-arginine acetate, 2-Iminopiperidine; 2,4-Diamino-6-hydroxy-pyrimidine; 5-chloro-1,3-dihydro-2H-benzimidazo 1-2-one (FR038251), 1,3(2H,4H)-isoquinoline-dione (FR038470) and 5-chloro-2,4(1H,3H)-quinazolonedione (FR191863).

11. The pharmaceutical composition according to claim 1, wherein the second therapeutically and/or prophylactically active substance is an opioid selected from the group consisting of heroin, fentanyl, morphine, oxycodone, hydrocodone, methadone, buprenorphine, pentazocine, butorphanol, dezocine, nalbuphine, Meperidine, normeperidine, hydromorphone, codeine, levorphanol and tramadol, BW373U86, CP 55,940 and SNC-121, and therapeutically active derivatives or metabolites thereof.

12. The pharmaceutical composition according to claim 1, wherein the second therapeutically and/or prophylactically active substance is a vanilloid receptor antagonist selected from the group consisting of Arvanil, Isovelleral, Olvanil, 5′-Iodoresiniferatoxin, Phorbol 12,13-didecanoate 20-homovanillate, Phorbol 12,13-dinonanoate 20-homovanillate, SB-366791, Scutigeral and Anti-Vanilloid Receptor-Like Protein 1, and therapeutically active derivatives thereof.

13. The pharmaceutical composition according to claim 1, wherein the second therapeutically and/or prophylactically active substance is 6-(5-carboxy methyl-hexyloxy)-2,2-dimethyl-hexanoic acid and analogues thereof as disclosed in WO 04/017952 and WO 03/003664.

14. The pharmaceutical composition according to claim 1, wherein the second therapeutically and/or prophylactically active substance is a neuroleptics agents defined by its ability to inhibit dopamine nerve transmission in the frontal lobes and being selected from the group consisting of Fluphenazine, prochlorperazine, Trifluoperazine, Perphenazine, Chlorpromazine, Thioridazine, mesoridazine besylate; thiothixine; haloperidol; olanzapine; molindone, loxapine, pimozide, clozapine, risperidone, quetiapine, Chlorprothixene, droperidol, Promethazine, Amitriptyline, ziprasidone, metoclopramid, and pharmaceutically acceptable salts or esters of any of the listed compounds.

15. The pharmaceutical composition according to claim 1, wherein the strontium containing compound and the second therapeutically and/or prophylactically active substance are contained in a single composition.

16. The pharmaceutical composition according to claim 1 wherein the strontium containing compound and the second therapeutically and/or prophylactically active substance are contained in a kit comprising a first and a second container, the first container comprising the strontium containing compound and the second container comprising the second therapeutically and/or prophylactically active substance.

17. The pharmaceutical composition according to claim 16 comprising instructions for substantially simultaneous or sequential administration of the strontium containing compound and the second therapeutically and/or prophylactically active substance.

18. The pharmaceutical composition according to claim 1 in the form of a tablet.

19. The pharmaceutical composition according to claim 18, wherein the tablet is coated with a coating that enables release of at least part of the strontium containing compound and/or the second therapeutically and/or prophylactically active substance salt in the proximal part of the small intestine.

20. The pharmaceutical composition according to claim 18, wherein the tablet has a shape that makes it easy and convenient for a patient to swallow.

21. The pharmaceutical composition according to claim 18, wherein the tablet has a rounded or a rod-like shape without any sharp edges.

22. The pharmaceutical composition according to claim 18, wherein the tablet is designed to be divided into two or more parts.

23. A method for improving pain management of an animal, the method comprising administration to an animal in need thereof of a effective amount of a strontium containing compound and a second therapeutically and/or prophylactically active substance selected from the group consisting of analgesic agents, anti-inflammatory agents and palliative agents.

24. The method according to claim 23, wherein the pain is
osteoarthritic pain,
rheumatoid arthritic pain,
juvenile chronic arthritis associated pain,
juvenile idiopathic arthritis associated pain,
Spondyloarthropathies
associated pain,
pain associated with psoriatic arthritis,
gout pain,
pain associated with pseudogout (pyrophosphate arthritis),
pain associated with systemic lupus erythematosus (SLE),
pain associated with systemic sclerosis (scleroderma),
pain associated with Behcet's disease,
pain associated with relapsing polychondritis,
pain associated with adult Still's disease,
pain associated with transient regional osteoporosis,
pain associated with neuropathic arthropathy,
pain associated with sarcoidosis,
arthritic pain,
rheumatic pain,
joint pain,
osteoarthritic joint pain,
rheumatoid arthritic joint pain,
juvenile chronic arthritis associated joint pain,
juvenile idiopathic arthritis associated joint pain,
Spondyloarthropathies
associated joint pain,
joint pain associated with psoriatic arthritis,
gout joint pain,
joint pain associated with pseudogout (pyrophosphate arthritis),
joint pain associated with systemic lupus erythematosus (SLE),
joint pain associated with systemic sclerosis (scleroderma),
joint pain associated with Behçet's disease,
joint pain associated with relapsing polychondritis,
joint pain associated with adult Still's disease,
joint pain associated with transient regional osteoporosis,
joint pain associated with neuropathic arthropathy,
joint pain associated with sarcoidosis,
arthritic joint pain,
rheumatic joint pain,
acute pain,
acute joint pain,
chronic pain,
chronic joint pain,
inflammatory pain,
inflammatory joint pain,
mechanical pain,
mechanical joint pain,
pain associated with the fibromyalgia syndrome (FMS),
pain associated with polymyalgia rheumatica,
monarticular joint pain,
polyarticular joint pain,
nociceptiv pain,
neuropathic pain,
psychogenous pain.
pain of unknown etiology,
pain mediated by IL-6, IL-6 soluble receptor, or IL-6 receptor,
pain associated with a surgical procedure in a patient with a clinical diagnosis of OA,
dental pain,
pain associated with a surgical procedure and or other medical intervention,
bone cancer pain,
neuropathic pain,
pain associated with migraine,
pain like static allodynia,
pain like dynamic allodynia,
pain associated with Crohn's disease and/or
headache pain.

25. The method according to claim 23 for alleviating pain other than joint pain, osteoarthritic pain, rheumatoid arthritic pain, and inflammatory joint pain, wherein the pain is pain mediated by IL-6, IL-6sR, or IL-6 receptor.

26. The method according to claim 23 for alleviating pain, wherein the pain is mediated by a protein or protein and its receptor selected from: oncostatin-M, oncostatin-M and oncostatin-M receptor, leukemia inhibitor factor (“LIF”), LIF and leukemia inhibitor factor receptor (“LIFR”), interleukin-1 (“IL-1”), and interleukin-1 receptor (“IL1 r”).

27. The method according to claim 23 for alleviating pain other than joint pain, osteoarthritic pain, rheumatoid arthritic pain, and inflammatory joint pain, wherein the pain is pain mediated by endothelin.

28. A method for treating an animal with a diagnosis of osteoarthritis (OA) with the aim of delaying disease progression of OA, the method comprising administration to an animal in need thereof an effective amount of a strontium containing compound and a second therapeutically and/or prophylactically active substance selected from the group consisting of analgesic agents, anti-inflammatory agents and palliative agents.

29. A method for treating an animal with a diagnosis of rheumatoid arthritis (RA) with the aim of delaying disease progression of RA, the method comprising administration to an animal in need thereof an effective amount of a strontium containing compound and a second therapeutically and/or prophylactically active substance selected from the group consisting of analgesic agents, anti-inflammatory agents and palliative agents.

30. (canceled)

31. The method according to claim 23, wherein the strontium containing compound and the second therapeutically and/or prophylactically active substance are contained in a single composition.

32. The method according to claim 23, wherein the strontium containing compound is administered in a daily dose of from about 100 to about 2000 mg ionic strontium.

33. The method according to claim 23, wherein the strontium containing compound is administered in combination with acetyl salicylic acid (ASA) and the daily dose of ASA is in a range of from about 1 to about 3000 mg/day.

34. The method according to claim 23, wherein the second therapeutically and/or prophylactically active substance is a selective COX-2 inhibitor that is administered in any of the following dose ranges: Rofecoxib: 10-50 mg/day, Valdecoxib: 5-20 mg/day, Celecoxib: 100-500 mg/day, Etoricoxib: 25-150 mg/day, Lumiracoxib: 100-500 mg/day, Parecoxib: 20-200 mg/day, Licofelone: 100-1000 mg/day

35. The method according to claim 23, wherein the second therapeutically and/or prophylactically active substance is an NSAID that is administered in any of the following dose ranges: meloxicam: 5-20 mg/day, piroxicam 10-30 mg/day, Naproxen: 500-1500 mg/day, Dexibuprofen: 500-1600 mg/day, Ibuprofen: 1000-3200 mg/day, Salsalate: 1000-3000 mg/day.

36. The method according to claim 23, wherein the second therapeutically and/or prophylactically active substance is ClNOD AZD3582 that is administered in daily dose of 200-2000 mg/day.

37. The method according to claim 23, wherein the animal is a human.

38. The method according to claim 23, wherein the animal is a domestic animal.

39. The pharmaceutical composition according to claim 5, wherein the second therapeutically and/or prophylactically active substance is a propionic acids which is naproxen, flurbiprofen, fenoprofen, ketoprofen or ibuprofen.

40. The pharmaceutical composition according to claim 5, wherein the second therapeutically and/or prophylactically active substance is a fenamate which is mefenamic acid.

41. The pharmaceutical composition according to claim 5, wherein the second therapeutically and/or prophylactically active substance is a pyrazolone which is phenylbutazone.

42. The pharmaceutical composition according to claim 5, wherein the second therapeutically and/or prophylactically active substance is a salicylate which is aspirin.

43. The pharmaceutical composition of claim 10, wherein the second therapeutically and/or prophylactically active substance is an isothiourea derivative which is S-Methylisothiourea, S-Ethylisothiourea, S-Isopropylisothiourea, or S-(2-Aminoethyl)-isothiourea.

44. The pharmaceutical composition according to claim 19, wherein the coating enables release in the proximal part of the small intestine of at least 50% w/w of the total amount of the strontium containing compound and/or the second therapeutically and/or prophylactically active substance salt in the tablet.

45. The pharmaceutical composition according to claim 19, wherein the coating enables release in the proximal part of the small intestine of at least 60% w/w of the total amount of the strontium containing compound and/or the second therapeutically and/or prophylactically active substance salt in the tablet.

46. The pharmaceutical composition according to claim 19, wherein the coating enables release in the proximal part of the small intestine of at least 65% w/w of the total amount of the strontium containing compound and/or the second therapeutically and/or prophylactically active substance salt in the tablet.

47. The pharmaceutical composition according to claim 19, wherein the coating enables release in the proximal part of the small intestine of at least 70% w/w of the total amount of the strontium containing compound and/or the second therapeutically and/or prophylactically active substance salt in the tablet.

48. The pharmaceutical composition according to claim 19, wherein the coating enables release in the proximal part of the small intestine of at least 80% w/w of the total amount of the strontium containing compound and/or the second therapeutically and/or prophylactically active substance salt in the tablet.

49. The pharmaceutical composition according to claim 19, wherein the coating enables release in the proximal part of the small intestine of at least 90% w/w of the total amount of the strontium containing compound and/or the second therapeutically and/or prophylactically active substance salt in the tablet.

50. The method according to claim 23 wherein the single composition is in the form of a tablet.

51. The method according to claim 23, wherein the strontium containing compound and the second therapeutically and/or prophylactically active substance are contained in a kit comprising a first and a second container, the first container comprising the strontium containing compound and the second container comprising the second therapeutically and/or prophylactically active substance.

52. The method according to claim 33, wherein the daily dose of ASA is from about 75 to about 320 mg/day.

53. The method according to claim 38, wherein the domestic animal is a dog (canis familiaris), cat (felix domesticus), cow (bos Taurus), horse (equus caballus), donkey or pig (sus scrofa).
